(19) United States
(12) Patent Application Publication
(10) Pub. No.: US 2005/0027227 A1
(43) Pub. Date: Feb. 3, 2005

(54) Disposable, water resistant cover for medical applications

(76) Inventors: Cynthia Dumas, Dallas, TX (US); Richard S. Dietz, Chicago, IL (US)

Correspondence Address: Richard C. Litman;LITMAN LAW OFFICES, LTD., P.O. Box 15035, Arlington, VA, 22215, US

(21) Appl. No.: 10/825,331

(22) Filed: Apr. 16, 2004

Related U.S. Application Data
(63) Continuation-in-part of application No. 10/423,867, filed on Apr. 28, 2003.

(51) Int. Cl. A61F 13/00

(52) U.S. Cl. 602/41

(57) ABSTRACT

Disposable, water resistant covers having a pair of resilient bands at each open end, and an absorbent medium disposed between the pair of resilient bands. The bands provide a substantially watertight seal between the cover and the users skin surface. The absorbent medium prevents any moisture that manages to bypass one band to reach the other band. The covers are formed to cover any medical application such as bandages, casts, splints, etc., on a patient's head, neck, chest, waist, arms, legs, and feet to protect against water seepage when attending to regular personal hygiene.

### CROSS-REFERENCE TO RELATED APPLICATION

This application is a continuation-in-part of Ser. No. 10/423,867, filed Apr. 28, 2003.

### BACKGROUND OF THE INVENTION

1. Field of the Invention

The present invention relates generally to water resistant coverings. More specifically, the invention is a disposable, water repellant covering for patients recovering from injury or surgery, having a water tight seal including a water absorbing medium.

2. Description of the Related Art

The prior art of interest describes various waterproofing covers for injured areas of a body, but none discloses the present invention. The use of wound and cast covers is known in the prior art. More specifically, wound and cast covers heretofore devised and utilized are known to consist basically of familiar, expected and obvious structural configurations, notwithstanding the myriad of designs encompassed by the crowded prior art which have been developed for the fulfillment of countless objectives and requirements. Referring to FIG. 7 of the instant specification, a general example of a conventional prior art cover type is illustrated. FIG. 7 shows a boot-shaped cover 700, formed of a flexible material 702 that is generally impervious to water and most other liquids. The opening of the cover 700 has a securing strap 704 that is generally helically wrapped around the opening and the skin surface of the limb. The securing strap 704 has a fastening end 706 that is used to immovably secured the strap 704 upon itself, so as to create a liquid proof seal about the limb upon which the cover 700 is disposed. Other related art of interest will be discussed hereafter, however none of these cited references are perceived to be relative to the present invention.

U.S. Pat. No. 4,178,924, issued on Dec. 18, 1979, to Samuel H. Baxter describes an elongated tubular protective cover for a cast enclosed body limb portion is provided and at least one end of the protective cover is open and includes an elongated flexible seal band of water impervious material carried by one end of the cover and for wrapping about that end and the adjacent limb portion disposed outwardly of the cover in an edge overlapping spiral manner in order to form a fluid tight seal between the cover and the adjacent limb portion.

U.S. Patent Application Publication No. US 2001/0041853 A1, published on Nov. 15, 2001, to Robert W. South et al., describes a cast cover configured to cover a specific portion of a user's body such as a leg, an arm, the torso or head includes an integral, elastomeric band adjacent an open end to provide a water tight seal between the cover and user's body.

U.S. Pat. No. 5,817,038, issued on Oct. 6, 1998, to Beatrice M. Orange et al., describes a system for supporting and protecting wounds, incisions, transdermal procedural sites, and associated medical equipment from moisture and other contaminants.

U.S. Pat. No. 4,639,945, issued on Feb. 3, 1987, to John J. Betz, describes a shield to protect a limb which has been injured, is using an orthopedic device, bandage, or the like, or otherwise requires protection from water during bathing of patients or the like.

U.S. Pat. No. 5,063,919, issued on Nov. 12, 1991, to Doris C. Silverberg, describes a water proof member protects casts, splints, or other appliances or surgical dressings from water damage. A combination of a securing member and straps secure the member and provide a water proof seal between the water proof member and the wearer.

U.S. Pat. No. 5,455,970, issued on Oct. 10, 1995, to Ricardo b. Bance et al., describes an adjustable shower and rain cap is provided, which consists of a headband worn about a head of a person. A waterproof pliable crown is affixed to the headband, so as to extend over the head of the person at different changeable heights.

U.S. Pat. No. 4,562,834, issued on Jan. 7, 1986, to Norman Bates et al., describes a waterproof covering intended to be worn over a cast or bandage on an injured arm or leg. The covering comprises a generally tubular water impervious sleeve which is closed at one end and which has spaced cutting sites running from side to side along which the sleeve can be cut to different lengths.

U.S. Pat. No. 3,329,144, issued on Jul. 4, 1967, to Peter C. Liman, describes a cast protector for arms and legs.

U.S. Pat. No. 3,374,1203, issued on June 26, 0.1973, to Peter C. Liman, describes a protective covering for encasing an injured body limb and preventing water or other harmful contaminants from contacting a wound and bandaging, or a cast.

U.S. Pat. No. 3,785,374, issued on Jan. 15, 1974, to Herbert G. Lipson, describes an elongated waterproof, flexible bag having an opening at on end through which a cast-bound limb is inserted and subsequently sealed about the limb with an inflatable cuff.

U.S. Pat. No. 6,191,335, issued on Feb. 20, 2001, to Joseph W. Robinson, describes a wound dressing consisting of a backing layer of hydrophilic polyurethane, an apertured or net wound facing layer of polyurethane, and an intermediate absorbent layer comprising a cotton fiber support layer containing a polyacrylic acid based superabsorber material. The absorbent fiber material will form a gel upon contact with any moisture. The device is distinguishable for requiring cotton fibers and polyacrylic acid based superabsorber material backed by a hydrophilic polyurethane layer.

U.S. Pat. No. 5,715,841, issued on Feb. 10, 1998, to Leo J. Utecht, describes a personal protection apparatus with an adhesive useful for covering burn areas, wound areas and contaminated surfaces of various kinds. The barrier comprises layers of absorbent material that may be a gel, hydrogel, hydrophobic web, or natural or synthetic fibrous material. The devices are distinguishable for requiring various gels, hydrogels, hydrophobic webs, or synthetic fibrous materials.

U.S. Pat. No. 6,512,158 B1, issued on Jan. 28, 2003, to John A. Dobos describes a medical protective wrap device comprising a rectangular liquid impermeable and stretchable polymeric wrap that provides a watertight seal and barrier for wounds and seepage.

Sealing cuffs are provided on opposite edges with reduced stretchability relative to the sheet. A fastener is attached to the first terminal edge to secure the sheet to the body. The second terminal edge is fastened to the sheet after at least one wrapping around the body part. The devices are distinguishable for requiring a polymeric wrap without gelling seals.

U.S. Patent Application NO. 2002/0091347 A1, published on Jul. 11, 2002, for Thomas G. Eakin, describes a wound dressing composition comprising an inner covering release layer consisting of paper coated with either polyethylene or silicon, a dressing layer comprising a gelling layer of either carboxymethyl cellulose alone with starch or sticky polyisobutylene (optionally mixed with polyethylene), and an indicator layer comprising dots of a soluble dye mixed with sodium carboxymethyl cellulose or polyisobutylene. Moisture from the wound would cause the activation of the indicator layer. The wound dressing composition is distinguishable for requiring activation of dyes by leaking moisture.

U.S. Patent Application No. 2002/0156411 A1, published on Oct. 24, 2002, for Helge Ahrens et al., describes a dressing composition comprising a top layer of a transparent multilayered polyurethane film over an adhesive layer comprising a polyacrylate including a hydrocarb on tackifier added to enhance adhesion to the skin. The dressing is beveled from its center. The dressing composition is distinguishable for requiring a beveled shape, a multilayered polyurethane film, and an adhesive layer of a polyacrylate and a hydrocarbon tackifier.

U.S. Pat. No. 4,523,586, issued on Jun. 18, 1985, to Mark S. Couri, describes a protective cover for a limb or a cast comprising a flexible fabric bag having hook and loop fastening patches or snaps to close the bag about a limb tightly. The covers are distinguishable for requiring only fabric bags and fastening.

U.S. Pat. No. 5,048,122, issued on Sep. 17, 1991, to Deborah G. Prieur, describes a baby garment or creeper for shielding lines such as catheters to the infant patient comprising a close fitting soft cotton garment body having a zippered front portion. The garment is distinguishable for lacking waterproofing.

U.S. Pat. No. 5,063,919, issued on Nov. 12, 1991, to Doris C. Silverberg, describes a waterproof protective sleeve made of polyethylene-vinyl for protecting casts, splints or other surgical dressings on arms, legs, and individual fingers from water damage comprising a combination of a securing tubular polyethylene-vinyl bag member and numerous encircling straps, a non-skid end strip to secure the bag member with the elastic members, and to provide a waterproof seal between the member and the wearer. The devices are distinguishable for requiring numerous encircling straps, end strips, and fingers.

U.S. Pat. No. 5,152,741, issued on Oct. 6, 1992, to Frank G. Famio, describes a surgical female chest or bra dressing comprising a flexible band formed primarily of a stretchable material with free overlapping ends that engage between the breasts. The band includes differential support structure to provide more support for the sides of the user. The surgical chest dressing is distinguishable for its required bra structure and the lack of a plastic waterproof cover.

U.S. Pat. No. 5,257,956, issued on Nov. 2, 1993, to Carol J. Ewen, describes a post-mastectomy garment comprising a padded vest-like garment adapted for applying comforting pressure to the sites of breast removal and other tissues, and for holding pain relieving packages. The garment is distinguishable for being limited to a vest adapted to hold packages.

U.S. Pat. No. 5,395,302, issued on Mar. 7, 1995, to Rudolph P. Botha et al., describes a protective waterproof sheath for a bandaged injured limb or in a cast comprising a polyvinyl bag made by sealing two sheets to fit a hand and forearm. The sheath can have both ends open to fit an elbow or a knee. The end has a closing strap that is more plasticized than the bag, and attached with hook and loop fastening patches. The device is distinguishable for requiring straps to prevent wetting of the bandaged region and the lack of the inventive gelling seals.

U.S. Pat. No. 5,562,642, issued on Oct. 8, 1996, to James A. Smith et al., describes separately packaged applicator pads for topical delivery of two incompatible dermatological drugs comprising a plurality of rectangular compartmentalized applicator pads that may be exposed and sequentially applied to the afflicted skin area. The device is distinguishable for requiring a plurality of compartmentalized applicator pads.

U.S. Pat. No. 6,503,526 B1, issued on Jan. 7, 2003, to Duane G. Krzysik et al., describes absorbent article compositions containing natural fats, natural oils, sterols or sterol derivatives, emollients, solidifying agents, and viscosity enhancers. The absorbent article comprises an outer cover, a liquid permeable bodyside liner, an absorbent body, and the chemical composition. The articles are distinguishable for requiring the addition of a specific dissimilar chemical composition.

U.S. Pat. No. 6,511,467 B1, issued on Jan. 28, 2003, to Denise Reuss, describes a fabric drainage reservoir medical garment including one or more drainage reservoirs in pockets. The medical garments are distinguishable for requiring pockets to hold drainage reservoirs.

European Patent Application No. EP 1 184 039 A2, published on Mar. 6, 2002d, for Yoshihide Murakami et al., describes an adhesive tape or sheet for application skin made of an acrylic copolymer having a specific carboxylic acid ester, wherein the acrylic copolymer has a gel fraction adjusted to a specific range. The adhesive tape or sheet can be used for first aid adhesive plasters, surgical tapes, large adhesive plasters with a pad, dressing materials, and the like. The device is distinguishable for being limited to an adhesive tape or sheet requiring acrylic copolymers.

None of the above inventions and patents, taken either singly or in combination, is seen to describe the instant invention as claimed. Thus a disposable, water resistant cover for medical applications solving the aforementioned problems is desired.

### SUMMARY OF THE INVENTION

There is a need for economical disposable waterproof covers that will enable an injured person perform general person hygienic functions without contaminating or degrading a bandage or cast with cleansers, water, etc.

A disposable, water resistant cover for medical applications is herein disclosed. The cover for medical applications includes a film having a predefined form, such as an enclosed sleeve, or boot. Also, the predefined form includes a pullover top, a partial pullover top and shorts. The film is a flexible, liquid impervious material. And the form defines at least one substantially circular opening. A resilient band is attached to the film and is disposed so as to encircle each of the at least one circular opening. The resilient band includes a first and second elastic band having a predetermined distance therebetween. An absorbent medium is attached to the film, and disposed between the first and the second elastic bands, so as to also encircle each of the at least one circular opening. Upon placing the film over a medical application, the first and second elastic bands form a substantially watertight seal against a skin surface of a wearer, and the absorbent medium absorbs any moisture that invades the film by passing the first band, and prevents the moisture from passing the second band. The absorbent medium includes a first wicking layer, and a second moisture absorption material layer.

### BRIEF DESCRIPTION OF THE DRAWINGS

FIG. 1 is an environmental view of a disposable, water resistant cover over a cast according to the present invention.

FIG. 2A is an enlarged view of the segment portion A of the FIG. 1 illustrating the various critical parts of the cover.

FIG. 2B is cross-sectional view of the segment portion A of the FIG. 2A, along section arrows 2B-2B.

FIG. 3 is a perspective view of the invention as applied to an injured lower extremity according to the inventive cover.

FIG. 4 is an environmental front view of a cover for an upper torso according to the inventive cover.

FIG. 5 is an environmental front view of a partial cover for an upper torso, and a cover for a lower torso according to the invention.

FIG. 6 is an environmental side view of a cover for a head according to the invention.

FIG. 7 is an environmental perspective view of a conventional prior art type of cast cover.

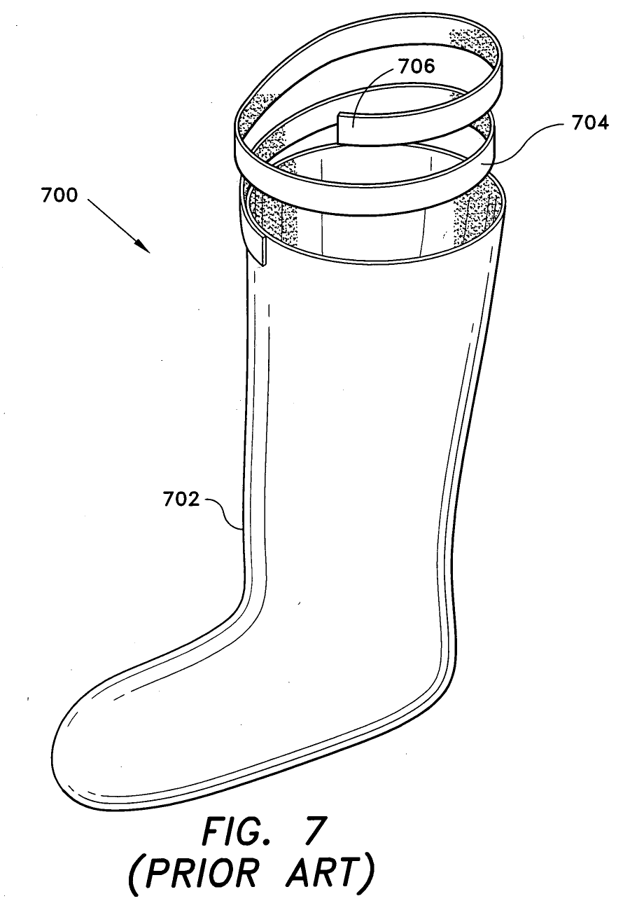

Similar reference characters denote corresponding features consistently throughout the attached drawings.

### DETAILED DESCRIPTION OF THE PREFERRED EMBODIMENTS

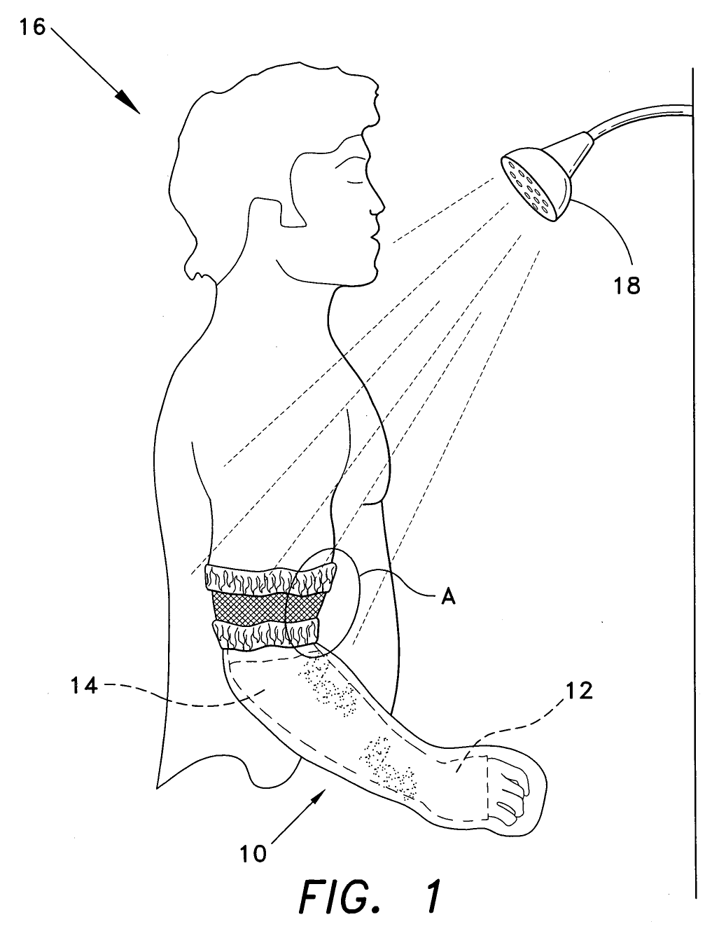

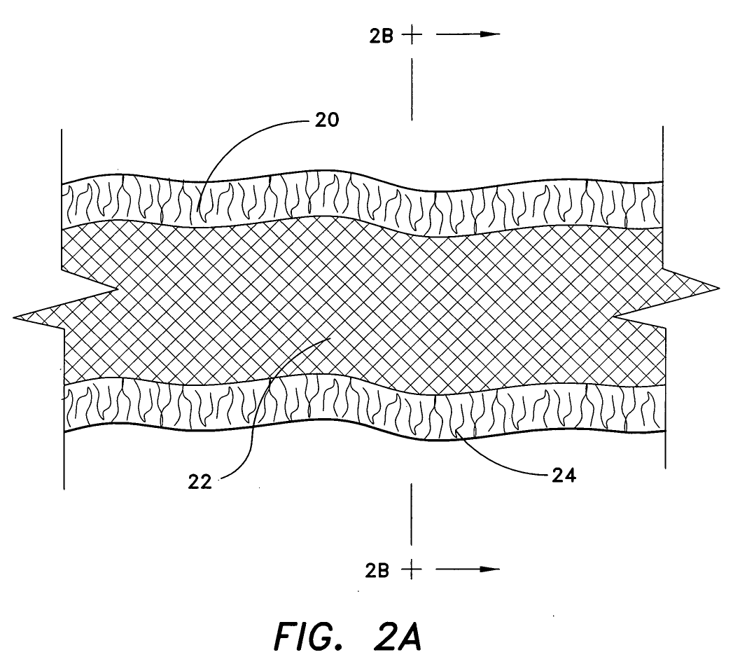

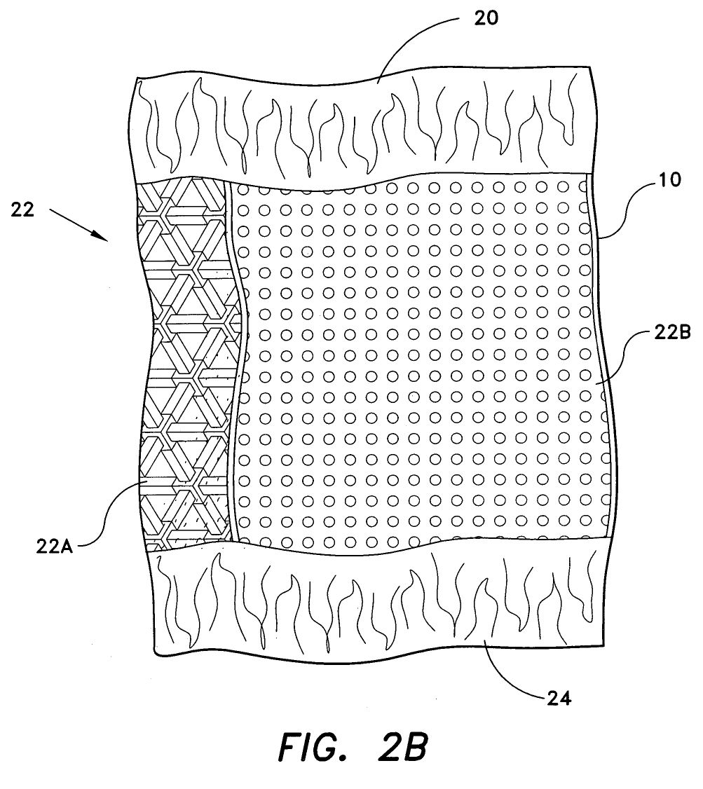

The present invention illustrated in FIG. 1 is directed to a flexible water resistant film or cover 10 for protecting a medical application 12 during a general hygienic procedure. The medical application 12 is generally referred to as a surgical bandage or cast (in phantom) on the forearm and hand 14 of a man 16, during the generally hygienic procedure, such as showering under a shower nozzle 18. The cover 10 is made of a flexible, durable, moisture impervious material, such as a plastic composition, or the like. The cover or film 10 has at least one opening for inserting a limb or body portion having the medical application thereon. FIGS. 2A and 2B illustrate the portion of FIG. 1 enclosed by the segment A. The film or cover 10 can have any color or be transparent. The elastic bands 22, 24 are designed and configured for providing a substantial constriction against the wearer's skin surface so to be watertight however, the constriction is generally not sufficient enough to decrease blood circulation beneath the surface of the skin.

As seen in FIG. 2A, a resilient band encircles the opening of the cover or film 10. The resilient band includes a first substantially elastic band 20, a second substantially elastic band 24, and an intermediate band 22, having a moisture absorbing property. s sewn to the covering 10 by hemming the edge of the covering 10 over the band 20. FIG. 2B is a cross-sectional view of the resilient band as viewed along 2B-2B. The film 10 is attached to the substantially elastic bands 20, 24, in a manner consistent with that of the waist, and leg bands of infant diapers, or adult incontinence pads. Between the bands 20, 24 is an intermediate moisture absorption layer 22. The moisture absorption layer 22 has two layers. The first layer 22A is a woven or non-woven fabric material having a moisture wicking property for causing moisture to adhere to its fibers. The second layer 22B is a granulated, or gelatin material that has a moisture absorption property, in that the layer 22B entraps the moisture adhered to by the wicking layer 22A. The second layer 22B is disposed adjacent to a portion of the film layer 10 between the elastic bands 20, 24. The layer 22B of the absorbent material, is generally a powder or granulated composition, or gelatin that absorbs moisture, entraps the moisture and prevents the moisture for traveling into the interior of the cover or film 10, thereby preventing the contamination of the medical application.

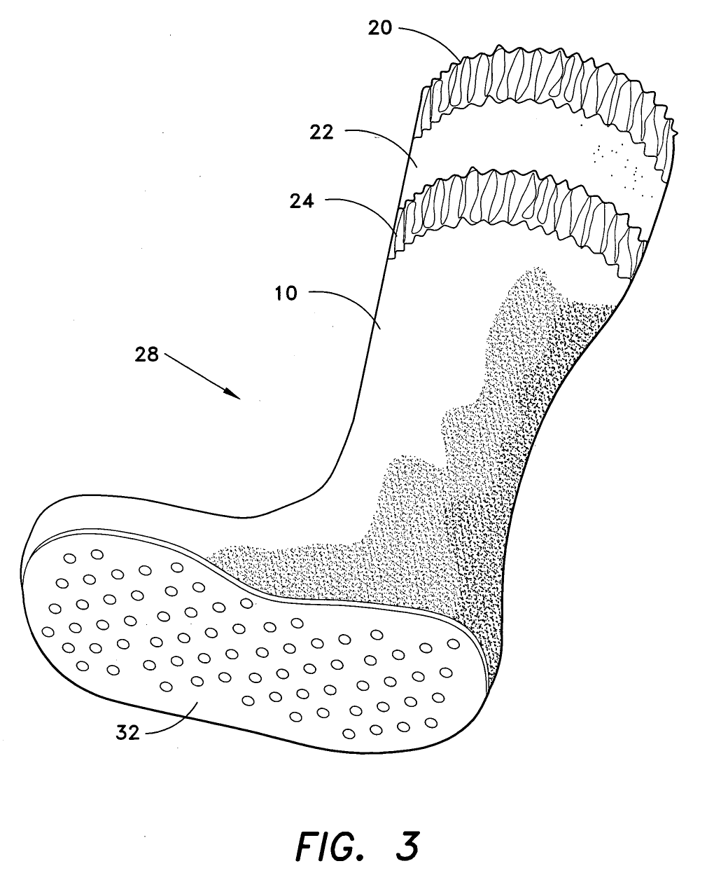

In FIG. 3, a cover 28 for a foot cast has a resilient band including the first elastic band 20, the moisture absorbing medium 22, and the second elastic band 24. In addition, a non-slip rubber sole 32 is optionally applied to the bottom or sole of the cover for protecting the cover 28, and for assisting the wearer's mobility by adding a frictional surface against slippage. The elastic bands 22, 24 are designed and configured for providing a substantial constriction against the wearer's skin surface so to be watertight however, the constriction is generally not sufficient enough to decrease blood circulation beneath the surface of the skin.

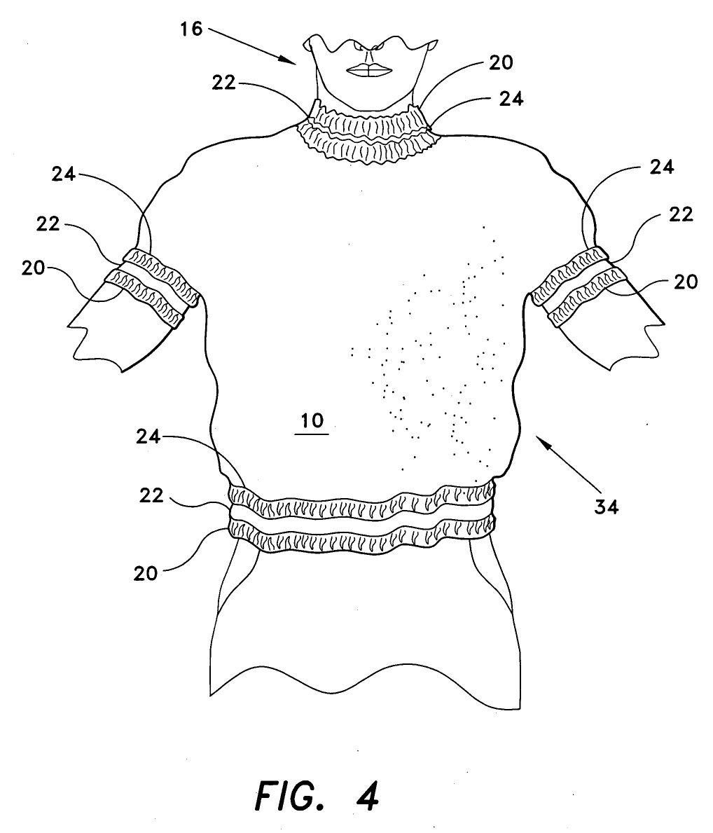

In FIG. 4, a full upper torso cover 34 similar to a pullover shirt, is illustrated showing the elastic bands 20 24, and the moisture absorption medium 22 for each of the at least one opening, the openings are found about the arms, neck and waist of the wearer 16. The absorption medium 22 includes the wicking layer 22A and the moisture absorption layer 22B as discussed in FIG. 2B above. The elastic bands 22, 24 are designed and configured for providing a substantial constriction against the wearer's skin surface so to be watertight however, the constriction is generally not sufficient enough to decrease blood circulation beneath the surface of the skin.

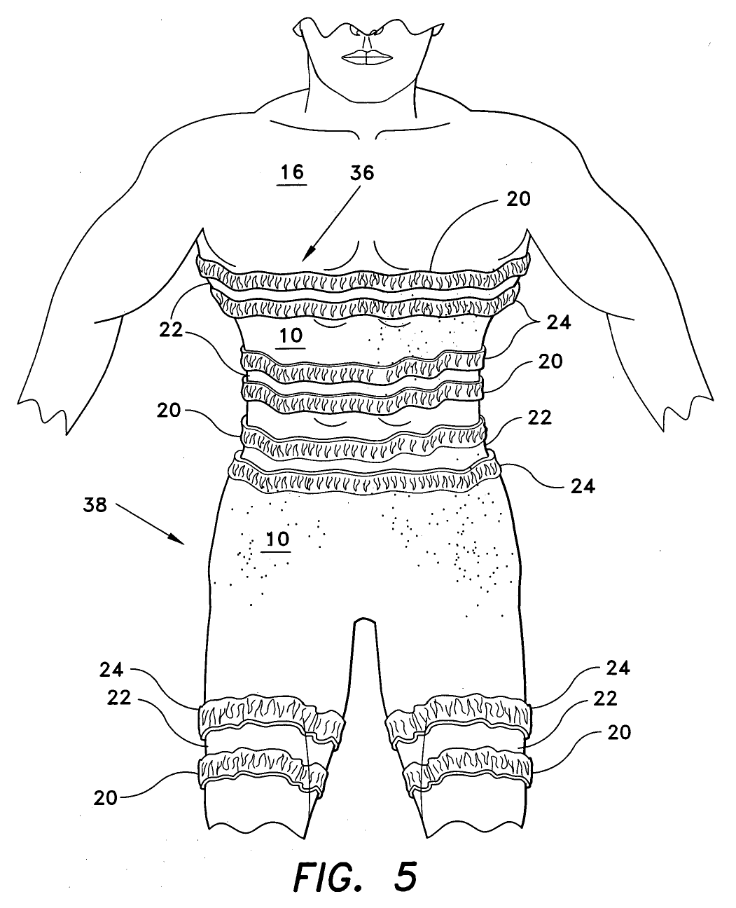

In FIG. 5, a partial upper torso cover 36 and a lower torso cover 38 are depicted with elastic bands 20 24, and the moisture absorption medium 22 for each of the at least one opening, the openings are found about the chest and waist (upper torso), and the waist, and thighs (lower torso) of the wearer 16. The absorption medium 22 includes the wicking layer 22A and the moisture absorption layer 22B as discussed in FIG. 2B above. The elastic bands 22, 24 are designed and configured for providing a substantial constriction against the wearer's skin surface so to be watertight however, the constriction is generally not sufficient enough to decrease blood circulation beneath the surface of the skin.

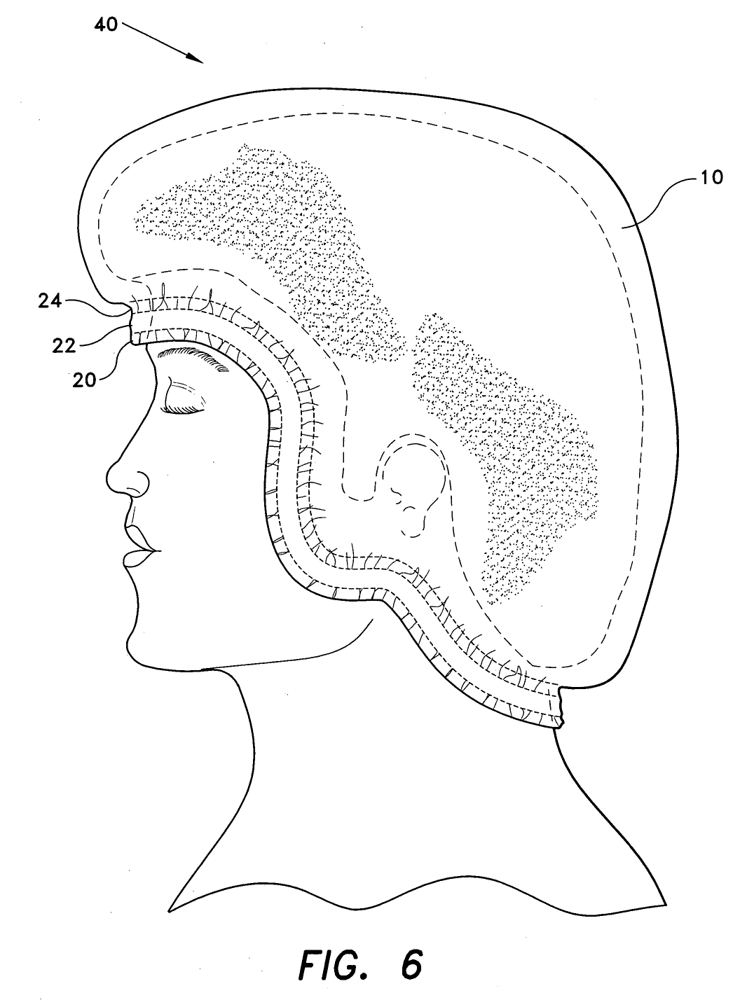

In FIG. 6, a head cover 40 similar to a shower cap, is illustrates the invention used by a wearer having a medical application (not shown) n a portion of the head. The head cover 40 shows the elastic bands 20 24, and the moisture absorption medium 22 surrounding the at least one opening, the opening is found about the head and neck of the wearer 16. The absorption medium 22 includes the wicking layer 22A and the moisture absorption layer 22B as discussed in FIG. 2B above. The elastic bands 22, 24 are designed and configured for providing a substantial constriction against the wearer's skin surface so to be watertight however, the constriction is generally not sufficient enough to decrease blood circulation beneath the surface of the skin.

It is herein disclosed that the cover 10 for medical applications includes a film having a predefined form, such as an enclosed sleeve, cap 40, or boot 28. Also, the predefined form includes a pullover top 34, a partial pullover top 36 and shorts 38. The film 10 is a flexible, liquid impervious material. And the form defines at least one substantially circular opening. A resilient band is attached to the film and is disposed so as to encircle each of the at least one circular opening. The resilient band includes a first 20 and second 24 elastic band having a predetermined

An absorbent medium 22 is attached to the film, and disposed between the first 20 and the second 24 elastic bands, so as to also encircle each of the at least one circular opening. Upon placing the film over a medical application, the first 20 and second 24 elastic bands form a substantially watertight seal against a skin surface of a wearer, and the absorbent medium 22 absorbs any moisture that invades the film by passing the first band 20, and prevents the moisture from passing by the second band 24. The absorbent medium 22 includes a first wicking layer 22A, and a second moisture absorption material layer 22B.

It is to be understood that the present invention is not limited to the embodiment***s described above, but encompasses any and all embodiments within the scope of the following claims.

1. A cover for medical applications comprising:
a film having a predefined form, said film formed of a flexible, liquid impervious material, said form defining at least one substantially circular opening;
a first resilient band attached to said film and disposed so as to encircle each of said at least one circular opening;
a second resilient band attached to said film and disposed at a predetermined distance from said first resilient band, so as to also encircle each of said at least one circular opening; and
an absorbent medium attached to said film, and disposed between said first and said second resilient bands, so as to also encircle each of said at least one circular opening;
wherein upon placing said film over a medical application, each said first and second resilient bands forming a substantially watertight seal against a skin surface, and said absorbent medium absorbs any moisture that invades said film by passing said first band, and prevents the moisture from passing said second band.

2. The cover according to claim 1, wherein said film is plastic.

3. The cover according to claim 1, wherein said film is adapted to protect a bandage.

4. The cover according to claim 1, wherein said film is adapted to protect a cast.

5. The cover according to claim 1, wherein said film is adapted to protect a body portion selected from the group consisting of the head, an arm, a chest, waist, a leg, and a foot.

6. The cover according to claim 5, wherein said film is for a head.

7. The cover according to claim 5, wherein said film is for an arm.

8. The cover according to claim 5, wherein said film is for a chest.

9. The cover according to claim 5, wherein said film is for a waist.

10. The cover according to claim 5, wherein said film is for a leg.

11. The cover according to claim 5, wherein said film is for a foot encased in a cast.

12. A cover for medical applications comprising:
a film having a predefined form, said film formed of a flexible, liquid impervious material, said form defining at least one substantially circular opening;
a resilient band attached to said film and disposed so as to encircle each of said at least one circular opening; and
an absorbent medium attached to said film, and disposed adjacent to said resilient band, so as to also encircle each of said at least one circular opening;
wherein upon placing said film over a medical application, each said resilient band forming a substantially watertight seal against a skin surface, and said absorbent medium absorbs any moisture that invades said film by passing said band, and prevents the moisture from entering said film.

13. The cover according to claim 12, wherein said resilient band includes a first and second substantially elastic bands, a said absorbent material is disposed between said first and second substantially elastic bands.

14. The cover according to claim 12, wherein said absorbent material includes a first layer for wicking moisture, and a second layer for entrapping the wicked moisture.

15. The cover according to claim 14, wherein said second layer includes a substance that substantially entraps moisture, thereby preventing contamination of the medical application.

16. The cover according to claim 15, wherein said substance is a granulated moisture absorbing medium.

17. The cover according to claim 15, wherein said substance is a gelatin moisture absorbing medium.
